(19) United States
(12) Patent Application Publication
(10) Pub. No.: US 2005/0054926 A1
(43) Pub. Date: Mar. 10, 2005

(54) Biometric user identification system and method for ultrasound imaging systems

(76) Inventor: Robert Lincoln, Bothell, WA (US)

Correspondence Address: ATL ULTRASOUND, P.O. BOX 3003, 22100 BOTHELL EVERETT HIGHWAY, BOTHELL, WA, 98041-3003, US

(21) Appl. No.: 10/913,087

(22) Filed: Aug. 5, 2004

Related U.S. Application Data
(60) Provisional application No. 60/501,097, filed on Sep. 8, 2003.

(51) Int. Cl. A61B 5/05

(52) U.S. Cl. 600/443

(57) ABSTRACT

An ultrasound imaging system (100) includes an imaging probe (20), an ultrasound signal path (40) coupled to the imaging probe (20), and an output device (16) for providing information about an examination conducted using the imaging system (100). The imaging system (100) also includes a biometric sensor (110) operable to generate biometric data that uniquely identifies an individual seeking to use the system (100). Biometric data for individuals who are registered to use the imaging system (100) are stored in the imaging system (100). An individual seeking to use the imaging system (100) accesses the biometric sensor (110) to enter biometric data. The entered biometric data is compared to the stored biometric data. The imaging system (100) is enabled in the event of a match. In the event of a match, the imaging system (100) may also be automatically configured using stored configuration settings for the individual, and images produced by the system (100) may be associated with stored user information for the individual.

This invention claims the benefit of Provisional U.S. Patent Application Ser. No. 60/501,097, filed Sep. 8, 2003.

### TECHNICAL FIELD

This invention relates to ultrasound imaging systems, and, more particularly, to a system and method for using biometric identification means to prevent unauthorized use of ultrasound imaging systems.

### BACKGROUND OF THE INVENTION

Ultrasound imaging systems widely used to obtain a variety of ultrasound images. The imaging systems may be used to scan different parts of the body and the same parts of the body using different techniques or imaging modalities. For example, the arm of a patient may be scanned by placing an ultrasound transducer against different surfaces of the arm to obtain images from different directions. Further, each image may be obtained by either keeping the ultrasound transducer stationery or scanning the transducer across the surface of the skin while the image is being obtained.

A typical ultrasound imaging system 10 of conventional design is shown in FIG. 1. The system 10 includes a chassis 12 containing most of the electronic circuitry for the system 10. The chassis 12 is mounted on a cart 14, and a display 16 is mounted on the chassis 12. An ultrasound imaging probe 20 is connected to the chassis 14 by a cable 24. The chassis 12 includes a keyboard and controls, generally indicated by reference numeral 28, for allowing a sonographer to enable the operation of the imaging system 10 and enter information about the patient or the type of examination that is being conducted.

In operation, the probe 20 is placed against the skin of a patient (not shown) and either held stationery or moved to acquire an image of blood or tissues beneath the skin. The image is presented on the display 16, and it may be recorded by a recorder (not shown) or data storage medium (not shown in FIG. 1). The system

10 may also record or print a report containing text and images. Data corresponding to the image may also be downloaded through a suitable data link, such as the Internet or a local area network.

It is desirable for the use of the ultrasound imaging system 10 to be restricted to authorized users for a variety of reasons. First, the imaging system 10 is capable of recording information about patients who have been examined using the imaging system 10, including alphanumeric text and ultrasound images. This patient information must be kept confidential, but the confidentiality would be compromised if unauthorized individuals could use the imaging system 10. Second, the quality of an ultrasound image depends to a large extent on the skill of the sonographer conducting the examination. A poor quality ultrasound image could make it difficult or impossible to detect a medically significant feature in the image, thus potentially leading to an incorrect diagnosis. It is therefore important to ensure that images are obtained using the ultrasound imaging system only by individually having the requisite degree of skill and training. Third, for accountability purposes, it is important to be able to determine the identity of the sonographer that obtained each of the images using the imaging system 10. For this reason, some information identifying the sonographer obtaining an image is normally included with a displayed or recorded image.

Various approaches have been used to restrict use of the ultrasound imaging system 10 to authorized users and to identify the sonographer obtaining each image using the system 10. The most basic technique is to require that the sonographer enter an identification and a unique password using the keyboard 28 before using the imaging system 10. The password may be, for example, either a set of alphanumeric characters memorized by the user or a pseudorandom number generated by a “key fob” or the like. The system 10 then compares the entered password with a stored list of authorized users. In the event of a match, the system 10 is enabled, and a record is made associating the name of the authorized user with each image obtained using the system 10.

Another technique is to encode authorization information in portable storage media, such as a Smartcard or magnetic strip in a badge or identification card. The system 10 includes a reader 30 for the storage media coupled to the chassis 12 by a cable 31. In the event of a match between the information in the storage media and an authorized list of users stored in the system 10, the system 10 is enabled, and a record is made associating the name of the authorized user with each image obtained using the system 10.

Each of these conventional techniques comes with its own set of disadvantages. The use of a password to provide authorization to use the imaging system 10 requires that the user either memorize a password (in addition to all of the other passwords that one needs to remember) or carry around a key fob or the like to provide a password. The security of this technique is also questionable. Passwords may be shared with unauthorized users, recorded in a manner that allows unauthorized users to determine the password, or discovered by unauthorized users while the password is being entered. The need to enter a password each time the imaging system 10 is used also slows down the rate at which ultrasound examinations can be conducted. The use of a key fob to provide the password presents additional problems, including the need to carry the key fob around to be able to use the imaging system 10 and compromised security if the key fob is lost or stolen. Similarly, the use of portable storage media to provide security also requires that authorized users be in possession of the storage media to use the imaging system 10, and security can also be compromised if the storage media is lost or stolen.

Another problem with the conventional imaging system 10 is the need to configure the system 10. Different sonographers may configure the system 10 in different ways even for the same type of ultrasound examination. Ultrasound imaging systems like the imaging system 10 are often shared by several sonographers. As a result, it is often necessary for each sonographer using the imaging system 10 to re-configure the system 10 each time it is used. The need to re-configure the system 10 before an ultrasound examination can be conducted can also limit the speed at which ultrasound examinations can be conducted using the system 10.

There is therefore a need for a system that more securely and easily allows authorized users access to the ultrasound imaging system, and that also allows the system to more quickly and easily be re-configured before it is used by each authorized user.

### SUMMARY OF THE INVENTION

An ultrasound imaging system includes a biometric sensor operable to generate biometric data that substantially uniquely identifies an individual seeking to use the ultrasound imaging system. Biometric data for individuals who are registered to use the ultrasound imaging system are stored in the imaging system. To use the system, an individual accesses the biometric sensor to enter biometric data. The entered biometric data are then compared to the stored biometric data. In the event of a match, the ultrasound imaging system is enabled so that the individual can use it to perform an ultrasound examination. When the ultrasound imaging system becomes enabled, the imaging system may also be automatically configured using stored configuration settings for the user, and reports, images and the like may be associated with user information for the individual that is also stored in the imaging system.

### BRIEF DESCRIPTION OF THE DRAWINGS

FIG. 1 is an isometric view of a conventional ultrasound imaging system.

FIG. 2 is an isometric view of an ultrasound imaging system according to one embodiment of the invention.

FIG. 3 is a block diagram of pertinent portions of the imaging system of FIG. 2.

FIG. 4 is a screen shot showing one technique that may be used to register authorized individuals to use the imaging system of FIGS. 2 and 3.

FIG. 5 is a flowchart showing the software executed by a processor in the imaging system of FIGS. 2 and 3 and showing the method in which the imaging system of FIGS. 2 and 3 operates to register authorized individuals.

FIG. 6 is a flowchart showing the software executed by a processor in the imaging system of FIGS. 2 and 3 and showing the method in which the imaging system of FIGS. 2 and 3 operates to determine if an individual is a registered user and to respond accordingly.

### DETAILED DESCRIPTION OF THE INVENTION

Embodiments of the present invention are directed to ultrasound imaging systems. Certain details are set forth below to provide a sufficient understanding of various embodiments of the invention. However, it will be clear to one skilled in the art that the invention may be practiced without these particular details. In other instances, well-known circuits, control signals, and timing protocols have not been shown in detail in order to avoid unnecessarily obscuring the invention.

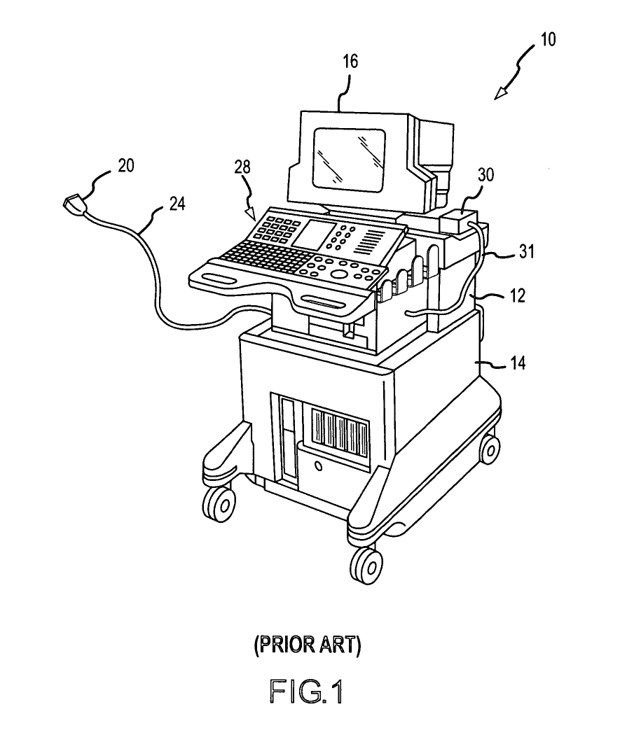

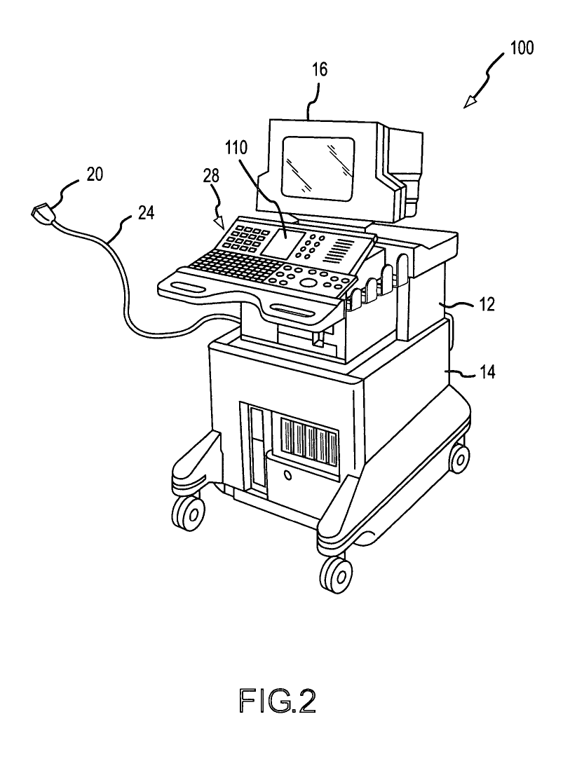

An ultrasound imaging system 100 in accordance with one embodiment of the invention is illustrated FIG. 2. The system 100 is physically identical to the system 10 shown in FIG. 1 in most respects. Therefore, the components of the system 100 have been provided with the same reference numerals as the components of the system 10, and an explanation of their function and operation will not be repeated. The system 100 is physically different from the system 10 in that it includes a biometric sensor 110 that can uniquely identify an individual attempting to use the system 100. The biometric sensor 110 outputs data that are unique to each individual attempting to use the system 100. As a result, unauthorized individuals are not able to use the system 100, and a record can be made associating a sonographer with each image or report made using the system 100. Additionally, the system 100 can be automatically re-configured for each sonographer.

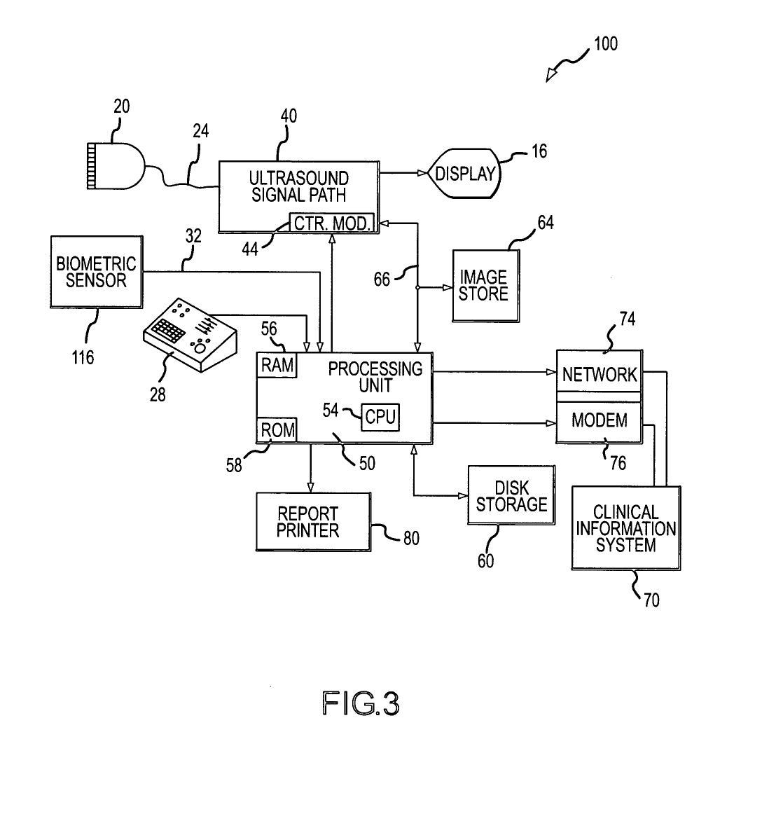

The electrical components in the ultrasound imaging system 100 are illustrated in greater detail in FIG. 3. The ultrasound imaging probe 20 is coupled through the cable 24 to an ultrasound signal path 40 of conventional design. As is well-known in the art, the ultrasound signal path 40 includes a transmitter (not shown) coupling electrical signals to the probe 20, an acquisition unit (not shown) that receives electrical signals from the probe 20 corresponding to ultrasound echoes, a signal processing unit (not shown) that processes the signals from the acquisition unit to perform a variety of functions, such as isolating returns from specific depths or isolating returns from blood flowing through vessels, and a scan converter (not shown) that converts the signals from the signal processing unit so that they are suitable for use by the display 16. The ultrasound signal path 40 also includes a control module 44 that interfaces with the processing unit 50 to control the operation of the above-described units. The ultrasound signal path 40 may, of course, contain components in addition to those described above, and, it suitable instances, some of the components described above may be omitted.

The processing unit 50 contains a number of components, including a central processor unit (“CPU”) 54, random access memory (“RAM”) 56, and read only memory (“ROM”) 58, to name a few. As is well-known in the art, the ROM 58 stores a program of instructions that are executed by the CPU 54, as well as initialization data for use by the CPU 54. The RAM 56 provides temporary storage of data and instructions for use by the CPU 54. The processing unit 50 interfaces with a mass storage device, such as a disk drive 60, for permanent storage of data, such as data corresponding to ultrasound images obtained by the system 10. However, such image data is initially stored in an image storage device 64 that is coupled to a signal path 66 extending between the ultrasound signal path 40 and the processing unit 50.

The processing unit 50 also interfaces with the keyboard and controls 28, which may be manipulated by the sonographer to configure the ultrasound imaging system and to enter information. The processing unit 50 preferably interfaces with a report printer 80 that provides reports containing text and one or more images.

The processing unit 50 is also coupled to the biometric sensor 10 via line 32. As explained in greater detail with reference to FIG. 6, biometric data from the biometric sensor 110 is compared to corresponding information in the table of registered users. In the event of a match, the system 100 is enabled, and a record is made associating the name of the registered user with each image obtained or each report generated using the system 100. Additionally, the system 100 may be automatically configured to the registered user based on a stored table of configuration settings that is accessed based on the data from the biometric sensor 110.

The biometric sensor 110 can be any sensor that provides information based on biological properties of an individual. These biological properties can be physical properties, chemical properties, electrical properties, or any other properties that are substantially unique to each individual. In one embodiment of the invention, the biometric sensor 110 is a fingerprint scanner that generates biometric data corresponding to the fingerprint of an individual in contact with the sensor 110. The biometric data from the sensor 110 is compared to corresponding data stored in the system for all fingerprints that have been registered with the system to determine if the individual being examined by the sensor 110 is authorized. Any suitable fingerprint scanner may be used. The fingerprint scanner may be a stand-alone fingerprint scanner such as a model U.are.U 2000 Fingerprint Sensor sold by DigitalPersona Inc. of Redwood City, Calif., or a fingerprint scanner integrated into the keyboard 28 such as a model U.are.U Fingerprint Keyboard, which is also available from DigitalPersona Inc.

In another embodiment of the invention, the biometric sensor is a retinal scanner (not shown) or an iris scanner (not shown). In other embodiments of the invention, the biometric sensor 110 is a speech recognition sensor (not shown) that is capable of uniquely recognizing the speech of a registered user. In another embodiment of the invention, the biometric sensor 110 is a face recognition sensor (not shown) that is capable of uniquely recognizing the face of a registered user. In other embodiment of the invention, the biometric sensor 110 is another type of sensor that is capable of uniquely identifying individuals.

Regardless of what type of biometric sensor 110 is used, corresponding data for all individuals who are registered with the system 100 are stored in the system 100 for comparison with the biometric data from the biometric sensor 110. As explained above, the table of registered users is preferably stored in the disk drive 60. The data are preferably stored in encrypted or other secure form, as is well known in the art, so that registered users cannot be added or deleted without proper authorization. Alternatively, the data for registered individuals may be stored elsewhere within or outside the system 100 as long as they can be accessed by the system 100. For example, the data for registered individuals may be stored in the clinical information system 70 and accessed through suitable means such as a local area network 74, a modem 76 or a wireless communication link (not shown). After the imaging system 100 has been enabled responsive to a match between entered biometric data and corresponding data for a registered user, the system 100 may be automatically set to a preferred configuration for the registered user.

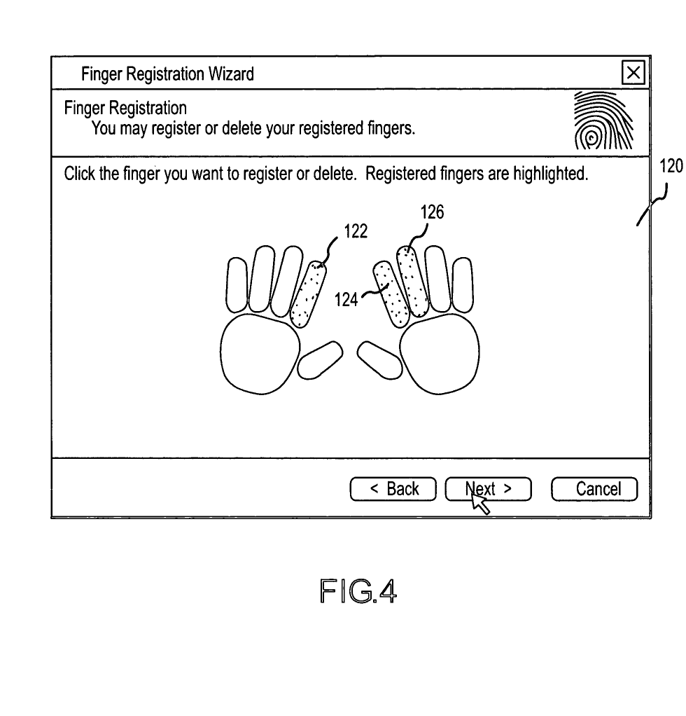

The manner in which authorized users are registered by the system 100 will now be explained with reference to FIG. 4. Once the system 100 is enabled by a system administrator, customer service representative or the like, an interactive screen display 120 is created that prompts the user to select the finger for biometric data that is to be registered in or deleted from the system 110. Fingers for which biometric data has already been registered, such as fingers 122, 124, 126, are shown highlighted in the display 120. Using a pointing device, such as a mouse, trackpad, trackball or other device, the user selects the finger to be added and then selects “next.” The user then accesses the biometric sensor 110, which produces biometric data that is associated with the selected finger and stored. The selected finger shown on the display 120 for the newly registered fingerprint then becomes highlighted. In the event biometric data for a registered fingerprint is to be deleted from the system 110, the user selects a previously highlighted finger then selects “next.”

The operation of the ultrasound imaging system 100 will now be explained with reference to FIG. 4. FIG. 4 comprises a flowchart showing the operation of the ultrasound imaging system 100, which is controlled by the processing unit 50 in accordance with a program stored in the ROM 58. The flowchart of FIG. 4 thus also constitutes an explanation of the software stored in the ROM 58 that is executed by the CPU 54.

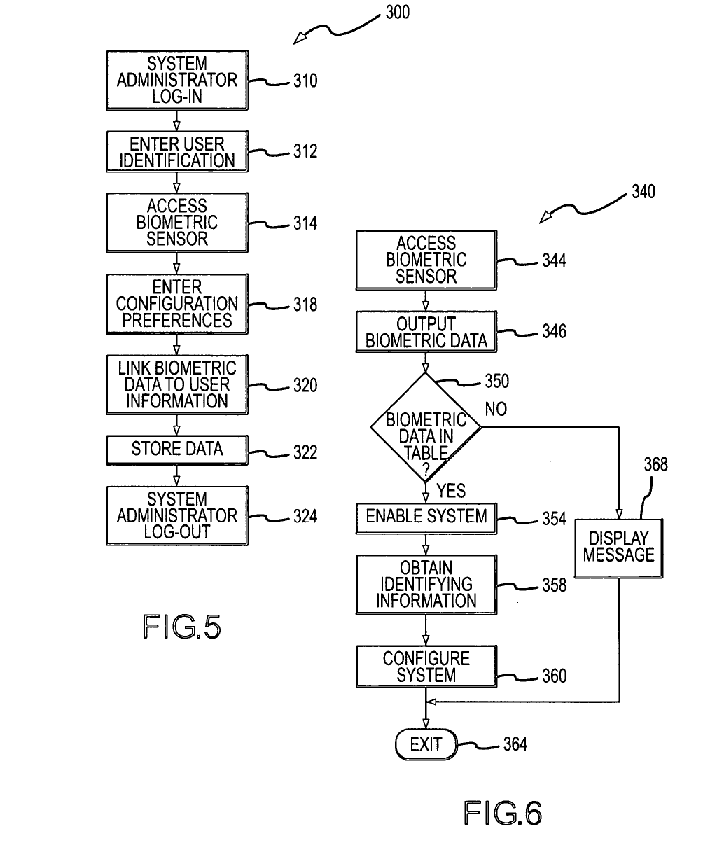

One embodiment of a procedure 300 for registering an authorized user to use the imaging system shown in FIG. 5. FIG. 5 comprises a flowchart showing the operation of the system 100 needed to carry out the procedure 300, which is controlled by the processing unit 50 in accordance with a program stored in the ROM 58. The flowchart of FIG. 5 thus also constitutes an explanation of the software stored in the ROM 58 that is executed by the CPU 54. The procedure is entered at 310 by a system administrator logging into the system 100 for purposes of registering an authorized user. Preferably using the keyboard 28, information is entered into the system 100 at step 312 that identifies the registered user, preferably by name or employee number. The registered user next accesses the biometric sensor 110 at step 314, thereby allowing the biometric sensor 110 to output biometric data uniquely identifying the registered user. In the event the biometric sensor 110 is a fingerprint scanner, this step 314 may be carried out as explained above with reference to FIG. 4. The user may enter his or her configuration preferences at step 318. These configuration preferences may be, for example, display preferences like typeface size and display color, or operating preferences like signal gain or frequency. In some embodiments, the preferences may be various combinations of configuration parameters so that, if one parameter is manually changed, the other parameter will automatically change. In any case, at step 320, the biometric data is linked to the user information that was entered at step 312 and the configuration preferences that were entered at step 318. The linked data are then stored at step 322. As mentioned above, the data is preferably stored at step 322 in a secure manner. The system administrator then logs out of the system 100 at step 324, thereby competing the procedure 300.

One embodiment of a procedure 340 for entering biometric data and verifying that the individual entering the data is a registered user is illustrated in FIG. 6. The procedure 340 is entered at step 344 by the individual accessing the biometric sensor 110. The biometric sensor 110 then produces biometric data at step 346 that uniquely identifies the individual. This entered biometric data are then compared to a table of corresponding data for registered users at step 350. In the event of a match, the system 100 is enabled at step 354. Additionally, the stored name, employee number, etc. linked to the stored biometric data for the individual is obtained at step 358 so that it can appear or otherwise be associated with any images, data or reports produced by the imaging system 100 while it is being used by the registered user. Finally, the imaging system 100 may be configured to the preferences of the individual at step 360 using the stored preferences that are linked to the stored biometric data for the individual. The procedure then exits at step 364. In the event no match is found at step 350 between the entered biometric data and the data in the table of registered users, an appropriate message is displayed at step 368 before exiting through step 364.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. An ultrasound imaging system (100), comprising:
an ultrasound imaging probe (20);
an ultrasound signal path (40) coupled to the ultrasound imaging probe (20);
an output device (16) for providing information about an ultrasound examination conducted by the ultrasound imaging system (100);
a biometric sensor (110) operable to generate biometric data that substantially uniquely identifies an individual seeking to use the ultrasound imaging system (100);
a storage device (60) storing biometric data for at least one individual who is registered to use the ultrasound imaging system (100); and
a processor (50) coupled to the signal path (40), the output device (16), the data entry device (28) and the storage device (60), the processor (50) being operable to compare the biometric data from the biometric sensor (110) to the stored biometric data, and, in the event of a match, enable the individual to use the ultrasound imaging system (110).

2. The ultrasound imaging system (100) of claim 1, wherein the storage device (60) storing the biometric data comprises a mass storage device (60) included in the ultrasound imaging system (100).

3. The ultrasound imaging system (100) of claim 2, wherein the mass storage device (60) included in the ultrasound imaging system (100) comprises a disk drive (60) included in the ultrasound imaging system (60).

4. The ultrasound imaging system (100) of claim 1, further comprising an input device (28) for entering configuration settings into the ultrasound imaging system (100), and wherein the processor (50) is further operable to store the entered configuration settings, and, when enabling the individual to use the ultrasound imaging system (100), retrieve the configuration settings from storage and automatically configure the ultrasound imaging system (100) according to the configuration settings.

5. The ultrasound imaging system (100) of claim 4 wherein the processor (50) is operable to automatically configure the ultrasound imaging system (100) according to combinations of configuration settings so that, if one configuration setting is manually changed, another configuration setting will automatically change.

6. The ultrasound imaging system (100) of claim 1, further comprising an input device (28) for entering user information about individuals who are registered to use the ultrasound imaging system (100), and wherein the processor (50) is further operable to store the entered user information, and, when enabling the individual to use the ultrasound imaging system (100), retrieve the user information from storage and associate the retrieved user information with information about the ultrasound examination provided by the output device (16).

7. The ultrasound imaging system (100) of claim 1 wherein the output device (16) comprises a display device (16).

8. The ultrasound imaging system (100) of claim 1 wherein the output device (16) comprises a report generator.

9. The ultrasound imaging system (100) of claim 1 wherein the storage device (60) storing the biometric data for at least one individual who is registered to use the ultrasound imaging system (100) comprises a clinical information system that is physically separate from the other components of the ultrasound imaging system (100).

10. The ultrasound imaging system (100) of claim 1 wherein the biometric sensor (110) comprises a fingerprint scanner that is operable to generate biometric data from the fingerprint of the individual seeking to use the ultrasound imaging system (100).

11. The ultrasound imaging system (100) of claim 1 wherein the biometric sensor (110) comprises an eye scanner that is operable to generate biometric data from the retina or iris pattern of the individual seeking to use the ultrasound imaging system (100).

12. The ultrasound imaging system of claim 1 wherein the biometric sensor (110) comprises a speech recognition sensor that is operable to generate biometric data from the speech of the individual seeking to use the ultrasound imaging system (100).

13. The ultrasound imaging system (100) of claim 1 wherein the biometric sensor (110) comprises a face recognition scanner (not shown) that is operable to generate biometric data from the face of the individual seeking to use the ultrasound imaging system (100).

14. The ultrasound imaging system (100) of claim 1 wherein the biometric sensor (110) comprises a chemical sensor that is operable to generate biometric data from a chemical property of the individual seeking to use the ultrasound imaging system (100).

15. The ultrasound imaging system (100) of claim 1 wherein the biometric sensor (110) comprises an electrical sensor that is operable to generate biometric data from an electrical property of the individual seeking to use the ultrasound imaging system (100).

16. A method of safeguarding an ultrasound imaging system (100) from unauthorized access, the method comprising:
storing a table of biometric data from registered users who are authorized to use the ultrasound imaging system (100);
entering biometric data into the ultrasound imaging system (100) from an individual who is attempting to obtain access to the ultrasound imaging system (100);
using the ultrasound imaging system (100) to compare the entered biometric data to the biometric data stored in the table; and
in the event of a match between the entered biometric data and the biometric data stored in the table, enabling the individual to use the ultrasound imaging system (100).

17. The method of claim 16 wherein the act of entering biometric data into the ultrasound imaging system (100) comprises accessing a fingerprint scanner (110) so that the fingerprint scanner (110) provides biometric data corresponding to a fingerprint of the individual.

18. The method of claim 16 wherein the act of entering biometric data into the ultrasound imaging system (100) comprises accessing an eye scanner so that the eye scanner provides biometric data corresponding to the retina or iris pattern of the individual.

19. The method of claim 16 wherein the act of entering biometric data into the ultrasound imaging system (100) comprises accessing a speech sensor so that the speech sensor provides biometric data corresponding to the speech of the individual.

20. The method of claim 16 wherein the act of entering biometric data into the ultrasound imaging system (100) comprises accessing a face recognition sensor so that the fingerprint scanner (110) provides biometric data corresponding to the face of the individual.

21. The method of claim 16 wherein the act of storing a table of biometric data from registered users who are authorized to use the ultrasound imaging system (100) comprises storing the table of biometric data in a mass storage device that is physically a part of the ultrasound imaging system (100).

22. The method of claim 16 wherein the act of storing a table of biometric data from registered users who are authorized to use the ultrasound imaging system (100) comprises storing the table of biometric data at a location remote from the ultrasound imaging system (100).

23. The method of claim 16, further comprising:
entering configuration settings into the ultrasound imaging system (100) for a plurality of the registered users;
storing the entered configuration settings;
when enabling the individual to use the ultrasound imaging system (100), retrieving the stored configuration settings for the individual; and
automatically configuring the ultrasound imaging system (100) according to the retrieved configuration settings.

24. The method of claim 16, further comprising:
entering user information into the ultrasound imaging system (100) for a plurality of the registered users;
storing the entered user information;
when enabling the individual to use the ultrasound imaging system (100), retrieving the stored user information for the individual; and
associating the retrieved user information with information about the ultrasound examination provided by the ultrasound imaging system (100).
